(19) United States
(12) Patent Application Publication
(10) Pub. No.: US 2022/0084655 A1
(43) Pub. Date: Mar. 17, 2022

(54) INFORMATION PROCESSING APPARATUS, INFORMATION PROCESSING METHOD, AND SYSTEM

(71) Applicant: TOYOTA JIDOSHA KABUSHIKI KAISHA, Toyota-shi (JP)

(72) Inventors: Tomoya Matsubara, Seto-shi (JP); Ibuki Shimada, Miyoshi-shi (JP); Keisuke Shoji, Nagoya-shi (JP); Shuhei Yamamoto, Aichi-gun (JP); Hideo Hasegawa, Nagoya-shi (JP); Yurika Tanaka, Yokosuka-shi (JP); Satoshi Komamine, Nagoya-shi (JP)

(73) Assignee: TOYOTA JIDOSHA KABUSHIKI KAISHA, Toyota-shi (JP)

(21) Appl. No.: 17/474,638

(22) Filed: Sep. 14, 2021

(30) Foreign Application Priority Data
Sep. 17, 2020 (JP) 2020-156741

(51) Int. Cl. G16H 20/70; G16H 20/30; G06Q 10/10; G01C 21/00

(52) U.S. Cl. CPC G16H 20/70; G16H 20/30; G06Q 50/01; G01C 21/005; G06Q 10/1097

(57) ABSTRACT

A more appropriate proposal is made far an action to be performed by a user in a free time of his or her schedule. A controller is provided which performs: obtaining schedules of a user; and transmitting to a terminal of the user, based on a content of a first schedule among the schedules of the user, a proposal for an action of the user to change his or her mood in a first free time in which the user has no schedule immediately before the first schedule.

### CROSS REFERENCE TO THE RELATED APPLICATION

This application claims the benefit of Japanese Patent Application No. 2020-156741, filed on Sep. 17, 2020, which is hereby incorporated by reference herein in its entirety.

### BACKGROUND

### Technical Field

The present disclosure relates to an info ion processing apparatus, an information processing method, and a system.

### Description of the Related Art

There have been known a technology that can provide an appropriate service in a free time based on information about the schedules of a user (see, for example, Patent Literature 1).

### CITATION LIST

### Patent Literature

Patent Literature 1: Japanese Patent Application Laid-Open Publication 2015-195014.

### SUMMARY

An object of the present disclosure is to make a more appropriate proposal for an action to be performed by a user in a free time in his or her schedules.

One aspect of the present disclosure is directed to an information processing apparatus including a controller configured to perform:

obtaining schedules of a user; and

outputting, based on a content of a first schedule among the schedules of the user, a proposal for an action of the user to change his or her mood in a first free time in which the user has no schedule immediately before the first schedule.

Another aspect of the present disclosure is directed to an information processing method for causing a computer to perform;

obtaining schedules of a user; and

outputting, based on a content of a first schedule among the schedules of the user, a proposal for an action of the user to change his or her mood in a first free time in which the user has no schedule immediately before the first schedule.

A further aspect of the present disclosure is directed to a system including a server configured to perform:

obtaining schedules of a user; and

transmitting to a terminal of the user, based on a content of a first schedule among the schedules of the user, a proposal for an action of the user to change his or her mood in a first free time in which the user has no schedule immediately before the first schedule.

In addition, a still further aspect of the present disclosure is directed to a program causing a computer to perform the above-described method, or a storage medium storing the program in a non-transitory manner.

According to the present disclosure, it is possible to make a more appropriate proposal for an action to be performed by a user in a free time in his or her schedules.

### BRIEF DESCRIPTION OF THE DRAWINGS

FIG. 1 is a diagram illustrating a schematic configuration of a system according to an embodiment;

FIG. 2 is a block diagram schematically illustrating an example of a configuration of each of a user terminal and a server, which together constitute the system according to the embodiment;

FIG. 3 is a diagram illustrating an example of a functional configuration of the server;

FIG. 4 is a diagram illustrating an example of a table structure of a user information DB;

FIG. 5 is a diagram illustrating an example of a table structure of a schedule information DB;

FIG. 6 is a diagram illustrating an example of a functional configuration of the user terminal;

FIG. 7 is a flowchart of processing of transmitting user information from the user terminal to the server according to the embodiment;

FIG. 8 is a flowchart of processing of transmitting schedule information from the user terminal to the server according to the embodiment;

FIG. 9 is a flowchart of processing of the server transmitting an action proposal to the user terminal according to the embodiment;

FIG. 10 is a flowchart of processing performed by the user terminal in a first free time according to the embodiment.

### DESCRIPTION OF THE EMBODIMENTS

An information processing apparatus, which is one aspect of the present disclosure, includes a controller. The controller performs: obtaining schedules of a user and outputting, based on a content of a first schedule among the schedules of the user, a proposal for an action of the user to change his or her mood in a first free time in which the user has no schedule immediately before the first schedule. The user's schedules include, for example, information such as a start time, an end time, a place, a content and so on. That is, the schedules of the user include information that allows the user to determine when, where, and what to do. The schedule of the user may be managed by a terminal of the user or may be managed by a server or the like. The schedules of the user may include schedules for user's works.

The first schedule is a schedule that will be performed after the first, free time, and is a future schedule that has not yet been performed. The controller proposes an action of the user to change his or her mood in the first free time before the start time of the first schedule. This action includes a movement or an exercise. The movement or exercise includes, for example, walking, stretching on the spot, or moving to a predetermined place. The predetermined place referred to here is, for example, a place where the first schedule can be performed, such as a public facility, a restaurant, a park or the like, which exists within a distance in which the user can move in his or her free time. In the case of proposing to move to the predetermined place, it is also proposed to perform the first schedule in the predetermined place. The proposal may include a route of travel to the predetermined place. Also, in the case of proposing walking, a walking route may also be proposed. The walking route may be, for example, a route to the predetermined place or a route to return to a departure place. In the case of proposing a route to the predetermined place, the route may be proposed according to the length of the first free time, the degree of fatigue of the user, or the like. In addition, as another method, for example, a route may be proposed in which the user passes through a spot for which a predetermined number or more of social buttons have been pressed in a social networking service (SNS) or passes through a spot described in a message posted to the SNS in the past by the user.

Then, the controller makes a proposal according to the content of the first schedule when proposing to the user an action of changing his or her mood in the first free time. This proposal is transmitted to the terminal of the user. This proposal may be transmitted within the first free time, or when the user is performing a schedule prior to the first free time. Depending on the content of a schedule, it may be difficult to perform the schedule at the predetermined place. For example, for a TV conference or the like, equipment is required, so that the schedule cannot be performed in a place where the equipment is not available. In addition, for example, even in the case of performing a work whose content must not be known to a third party, it is not possible to do the work in a place where the third party is present. Therefore, the controller may determine whether or not the user is capable of performing the first schedule in the predetermined place.

For example, the controller may determine, based on the content of the first schedule, whether or not the user is capable of performing the first schedule in the predetermined place, and when the user is capable of performing the first schedule in the predetermined place, the controller may propose that the user move, to the predetermined place during the first free time, as the proposal for the action of the user to change his or her mood. That is, the controller may propose that the user perform the first, schedule in the predetermined place. In this way, in cases where the user is capable of performing the schedule in the predetermined place, the controller may propose that the user move to the predetermined place thereby enabling the user to change his or her mood.

In addition, for example, based on the content of the first schedule, the controller may determine whether or not the user is capable of performing the first schedule in the predetermined place, and in cases where the user is not capable of performing the first schedule in the predetermined place, the controller may propose that the user perform an exercise in the first free time, as the proposal for the action of the user to change his or her mood. The exercise may include walking, jogging, or stretching. As described above, in cases where the user cannot perform the first schedule in the predetermined place, it is possible to change the mood of the user by proposing an exercise to the user.

Hereinafter, embodiments of the present disclosure will be described based on the accompanying drawings. The configurations of the following embodiments are examples, and the present disclosure is not limited to the configurations of the embodiments. In addition, the following embodiments can be combined with one another as long as such combinations are possible and appropriate.

### First Embodiment

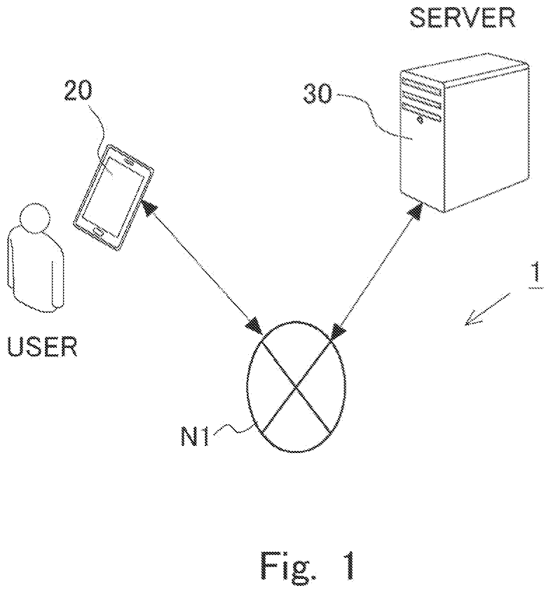

FIG. 1 is a diagram illustrating a schematic configuration of a system according to an embodiment. In the example of FIG. 1, the system 1 includes a user terminal 20 and a server 30. The user terminal 20 is a terminal that is used by a user. An application for managing schedules of the user is installed in the user terminal 20.

The system 1 makes a proposal for an action to be performed in a free time (first free time) until a next schedule (first schedule) according to the content of the first schedule. The proposal is, for example, a proposal for a walking route or a proposal for a place where the first schedule is carried out. For example, in cases where a schedule of the user is a work that can be done at an optional place (for example, in the case of a work of inputting characters to a personal computer), it may not be a problem to move the place where the work is done. In this case, for example, the user may move to a restaurant such as a cafeteria to work. By working in the cafeteria, the user can change his or her mood. Also, moving to the cafeteria can be a refreshing change of pace, either through exercise or by enjoying the view outside during the movement.

On the other hand, when the first schedule includes, for example, a TV conference or a WEB conference using two-way communication, it is difficult to work in a cafeteria around which a third party may be present. In addition, it is difficult to carry out a TV conference except in places where the necessary facilities are prepared. In such a case, when proposing an action according to the first schedule, it is proposed not to move to a place where a third party is present or a place where necessary facilities are not prepared, but to perform an exercise such as walking or stretching. For example, when a walk is proposed, a place where a TV conference can be held is set as the destination of the walk.

In addition, the proposal for movement may be made in accordance with the degree of fatigue of the user, the efficiency of work, the length of free time, the desire of the user, or the like. For example, the walking route may be determined according to the degree of fatigue of the user, the efficiency of work, the length of the free time, the desire of the user, or the like. For example, the number of pulses or breaths of the user may be detected as a parameter correlated with the degree of fatigue of the user, and the degree of fatigue of the user may be detected based on the number of pulses or breaths of the user. The degree of fatigue can be detected by using a known technique. Also, the number of pulses or breaths of the user can be detected by, for example, a sensor included in a wearable device. In addition, in a schedule immediately before the first free time (hereinafter, also referred to as an old schedule), the number of characters inputted to the user terminal 20 per unit time or the number of pages of documents viewed by the user terminal 20 per unit time may be detected as a parameter correlated with the degree of fatigue of the user, and when these numbers indicate a decreasing tendency, it may be determined that the efficiency of the work is decreasing. The number of characters inputted to the user terminal 20 per unit time or the number of pages of documents viewed by the user terminal 20 per unit time is obtained from, for example, word processor software or document viewing software installed in the user terminal 20.

Moreover, for example, in the old schedule, it may be determined that the degree of fatigue of the user is higher as the number of characters inputted to the user terminal 20 is larger or the distance of movement of a mouse is longer. Also, since some users may not like walking, a plurality of proposals may be made to the user as an action for the free time, so that, the user may select one of the proposals. In addition, the server 30 may determine a moving course (walking route) according to the length of the free time, or may also determine a moving course based on the preference of the user. For example, in cases where the messages posted by the user on a social networking service (SNS) includes a message about a specific place, the server 30 may determine whether or not it is possible for the user to move to that place within the free time, and if possible, it can propose a movement to the place. Further, if the user likes new things, a movement to a place that is a topic on the SNS may be proposed.

The user terminal 20 and the server 30 are connected to each other by a network N1. The network N1 is, for example, a worldwide public communication network such as the Internet, and a WAN (wide area network) or other communication networks may he adopted. Also, the network N1 may include a telephone communication network such as a mobile phone network or the like, or a wireless communication network such as Wi-Fi (registered trademark) or the like. Here, note that FIG. 1 illustrates one user terminal 20 by way of example, but there can be a plurality of user terminals 20.

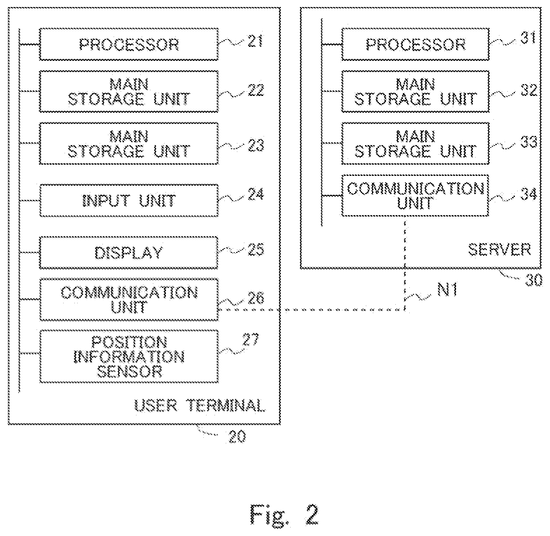

Hardware configurations and functional configurations of the user terminal 20 and the server 30 will be described based on FIG. 2. FIG. 2 is a block diagram schematically illustrating an example of a configuration of each of the user terminal 20 and the server 30, which together constitute the system 1 according to the present embodiment.

The server 30 has a configuration of a general computer. The server 30 includes a processor 31, a main storage unit 32, an auxiliary storage unit 33, and a communication unit 34. These components are connected to one another by means of a bus. The processor 31 is an example of a controller.

The processor 31 is a CPU (Central Processing Unit), DSP (Digital Signal Processor), or the like. The processor 31 controls the server 30 thereby to perform various information processing operations. The main storage unit 32 is a RAM (Random Access Memory), a ROM (Read Only Memory), or the like. The auxiliary storage unit 33 is an EPROM (Erasable Programmable ROM), a hard disk drive (HDD), a removable medium, or the like. The auxiliary storage unit 33 stores an operating system (OS), various programs, various tables, and the like. The processor 31 loads a program stored in the auxiliary storage unit 33 into a work area of the main storage unit 32 and executes the program, so that each component or the like is controlled through the execution of the program. As a result, the server 30 realizes functions that match predetermined purposes. The main storage unit 32 and the auxiliary storage unit 33 are computer readable recording media. Here, note that the server 30 may be a single computer or a plurality of computers that cooperate with one another. In addition, the information stored in the auxiliary storage unit 33 may be stored in the main storage unit 32. Also, the information stored in the main storage unit 32 may be stored in the auxiliary storage unit 33.

The communication unit 34 is a means or unit that communicates with the user terminal 20 via the network N1. The communication unit 34 is, for example, a LAN (Local Area Network) interface board, a wireless communication circuit for wireless communication, or the like. The LAN interface board or the wireless communication circuit is connected to the network N1.

Next, the user terminal 20 will be described. The user terminal 20 is a smart phone, a mobile phone, a tablet terminal, a personal information terminal, a wearable computer (such as a smart watch or the like), or a small computer such as a personal computer (PC). The user terminal 20 includes a processor 21, a main storage unit 22, an auxiliary storage unit 23, an input unit 24, a display 25, a communication unit 26, and a position information sensor 27. These components are connected to one another by means of a bus. The processor 21, the main storage unit 22 and the auxiliary storage unit 23 are the same as the processor 31, the main storage unit 32 and the auxiliary storage unit 33 of the server 30, respectively, and hence, the description thereof will be omitted.

The input unit 24 is a means or unit that receives an input operation performed by the user, and is, for example, a touch panel, a mouse, a keyboard, a push button, or the like. The display 25 is a means or unit for presenting information to the user, and is, for example, an LCD (Liquid Crystal Display), an EL (Electroluminescence) panel, or the like. The input, unit 24 and the display 25 may be configured as a single touch panel display, The communication unit 26 is a communication means or unit for connecting the user terminal 20 to the network N1. The communication unit 26 is, for example, a circuit for communicating with other devices (e.g., the server 30 and the like) via the network N1 by making use of a radio or wireless communication network such as a mobile communication service (e.g., a telephone communication network such as 5G (5th Generation), 4G (4th Generation), 3G (3rd Generation), LTE (Long Term Evolution) or the like), Wi-Fi (registered trademark), Bluetooth (registered trademark) or the like.

The position information sensor 27 obtains position information (e.g., latitude and longitude) of the user terminal 20 at predetermined intervals. The position information sensor 27 is, for example, a GPS (Global Positioning System) receiver unit, a wireless communication unit or the like. The information obtained by the position information sensor 27 is recorded, for example, in the auxiliary storage unit 23 or the like, and transmitted to the server 30.

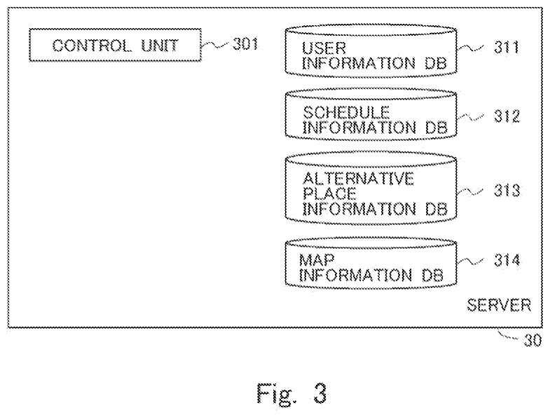

Next, the functions of the server 30 will be described. FIG. 3 is a view illustrating an example of a functional configuration of the server 30. The server 30 includes, as its functional components, a control unit 301, a user information DB 311, a schedule information DB 312, and an alternative place information DB 313, and a map information DB 314. The processor 31 of the server 30 executes the processing of the control unit 301 by a computer program on the main storage unit 32. The user information DB 311, the schedule information DB 312, the alternative place information DB 313, and the image information DB 314 are built by a program of a database management system (DBMS) that is executed by the processor 31 to manage data stored in the auxiliary storage unit 33. The user information DB 311, the schedule information DB 312, the alternative place information DB 313, and the map information DB 314 are, for example, relational databases. Here, note that any of the individual functional components of the server 30 or a part of the processing thereof may be executed by another computer connected to the network N1.

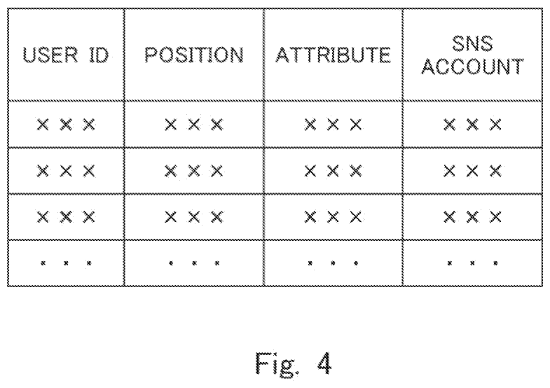

The control unit 301 obtains user information and schedule information corresponding to the user. The user information and the schedule information are, for example, inputted or entered by the user into the user terminal 20, and transmitted from the user terminal 20 to the server 30. Upon obtaining the user information, the control unit 301 stores the user information in the user information DB 311. The user information includes, for example, information about the position of the user, information about an attribute, of the user, or information about an SNS account of the user. FIG. 4 illustrates an example of a table structure of the user information DB 311. The user information table includes fields for user ID, position, attribute, and SNS account.

In the user ID field, identification information unique to each user (which may also be each user terminal 20) is inputted or entered. The user ID is assigned to each user or each user terminal 20 by the control unit 301. In the position field, the position information transmitted from each user terminal 20 is entered. In the attribute field, the attribute of each user is entered. The attribute of each user is information used as a reference when proposing an action of the user to change his or her mood in the first free time, The attribute of each user may be, for example, information about age, gender, hobby, preference, or the like. The hobby and preference of each user is, for example, information that can be used, for example, to determine whether the user likes exercise, dislikes exercise, likes to drive, likes quiet places, likes lively places, likes to look at flowers, or the like. The attribute of each user is, for example, information that each user has entered into his or her user terminal 20. As an alternative method, for example, the control unit 301 may obtain the attribute of a user by obtaining a message posted to the SNS by the user based on the SNS account of the user, and obtaining, based on this message, information about a place in which the user is interested. Here, note that the user information may be transmitted from each user terminal 20 at predetermined time intervals. Also, the attribute of each user may have been registered in advance via each user terminal 20.

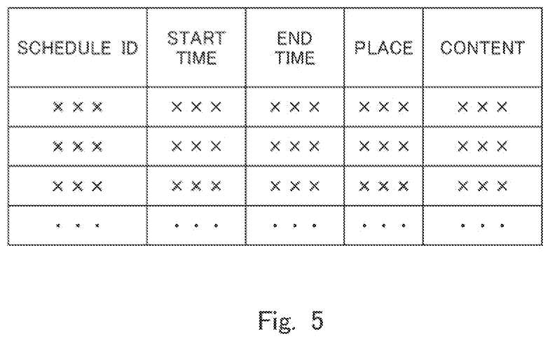

Upon obtaining the schedule information, the control unit 301 stores it in the schedule information DB 312 in association with a user ID. The schedule information includes a schedule ID for identifying each schedule, a start time, an end time, a place, and a content of the schedule. The schedule information is managed for each user. FIG. 5 is a diagram illustrating a table structure of the schedule information DB 312. The schedule information table has fields for schedule ID, start time, end time, place, and content.

In the schedule ID field, identification information unique to each schedule is entered. In the start time field, the start time of each schedule is entered. In the end time field, the end time of each schedule is entered. In the place field, information about the place in which each user works, and which is inputted when the user enters a schedule, is entered. Here, note that the information about the place entered in the place field may be information about the place on the assumption that each user does not move during the immediately preceding free time. In the content field, information about the content of each work is entered. In the content field, information is entered which enables each user to determine whether or not the place where his or her schedule is performed can be changed. The information entered in the content field may include, for example, whether or not to conduct a TV conference, whether or not each work requires confidentiality, or whether or not a device other than a user terminal 20 is required.

In addition, in the alternative place information DB 313, for example, information about places where work can be performed (hereinafter also referred to as alternative places) is entered. For example, information about restaurants, parks, public facilities, or the like is entered. This information includes, for example, information about the position of each place, business hours, and the content of each work that can be performed. The information about the content of each work that can be performed may include, for example, information capable of determining whether or not a TV conference can be held. The alternative place information is registered in advance by a user who manages the server 30 or a user who manages the alternative places information about, the congestion state of each alternative place may be entered in the alternative place information DB 313. In this case, information capable of determining whether or not there is a space in which each user works may be entered.

Moreover, map information, including map data and POI (Point of Interest) information such as texts and/or photographs that show the characteristics of each point on the map data, is stored in the map information DB 314. The map information 314 may be provided from other systems connected to the network N1 such as, for example, a GIS (Geographic Information System). The map data includes, for example, link data related to roads (links), node data related to node points, intersection data related to each intersection, search data for searching routes, facility data related to facilities, search data for searching points or locations, etc.

The control unit 301 proposes an action to be performed by the user in the first free time immediately before the first schedule based on the schedules of the user. This proposal is a proposal for changing the mood of the user, and is made based on the user information and schedule information. The action to be proposed may be determined based on, for example, the degree of fatigue of the user, the length of the first free time, or the hobby and preference of the user, in addition to the content of the first schedule.

As described above, the control unit 301 proposes an action based on the content of the first schedule. For example, if the work to be performed in the first schedule is a work that can also be performed in a public place, a movement to a restaurant may be proposed to the user. Note that the control unit 301 may propose, as a walking route, a route to a place where the first schedule is to be performed. The place where the first schedule is to be performed may be proposed as a place where a change in mood of the user is attained. On the other hand, if the content of the first schedule includes, for example, a TV conference, it is difficult to perform the schedule in a public place, and hence, an action such as walking or stretching on the spot may be proposed. For the walking, there may be generated and proposed a route to allow the user to return by the start time of the first schedule.

Further, the control unit 301 may propose an action to be performed in the first free time based on the degree of fatigue of the user. The degree of fatigue of the user may be determined by detecting a physical quantity correlated with the degree of fatigue of the user. As the physical quantity correlated with the degree of fatigue of the user, for example, a respiration rate or a heart rate of the user can be mentioned. In addition, it is also possible to determine the degree of fatigue of the user, for example, based on the content of the schedule (old schedule) immediately before the first free time. For example, it can be considered that the degree of fatigue of the user becomes higher according to the length of the old schedule. Also, for example, in the old schedule, it may be considered that the degree of fatigue of the user becomes higher as the number of characters entered into the user terminal 20 increases, or it may be considered that the degree of fatigue of the user becomes higher as the number of characters entered into the user terminal 20 per unit time increases. Moreover, for example, in cases where there is a decreasing tendency in the number of characters per unit time entered into the user terminal 20 in the old schedule, the higher the degree of decrease in the number of characters per unit time entered, the higher the degree of fatigue of the user may be considered.

The control unit 301 may quantify the degree of fatigue of the user, so that only when the degree of fatigue of the user exceeds a predetermined threshold value, the control unit 110 may propose an action in the first free time. That is, when the degree of fatigue of the user is low, no proposal may be made. In addition, the control unit 301 may propose an action according to the degree of fatigue of the user.

Furthermore, the control unit 301 may propose an action based on the length of the first free time. For example, the walking route may be determined according to the length of the first free time. In this case, for example, the walking route may be generated such that the longer the first free time, the longer the walking distance.

In addition, the control unit 301 may propose an action based on the hobby and preference of the user as an action to be performed during the first free time. For example, messages posted by the user to the SNS may be obtained, and what the user wants to do may be extracted from the messages, so that an action corresponding to what the user wants to do may be proposed. For example, in cases where a message posted to the SNS includes information about a store or a place to which the user wants to go, the user may be proposed or suggested to perform the first schedule at the store or place. Also, as an alternative method, for example, a place that the user is likely to prefer may be estimated from the movement history of the user in the past, free times. For this estimation, for example, machine learning may be used. As another method, for example, a movement to a place that is a topic in the SNS, such as a place where a predetermined number or more of social buttons have been pressed, may be proposed to the user.

When the control unit 301 generates a walking route or a route to an alternative place, for example, a place preferred by the user may be included on the route. For example, in eases where the attribute of the user stored in the user information DB 311 indicates that the user likes flowers, information about places where flowers are in bloom may be obtained based on the postings of the SNS, and a route passing through a place(s) where flowers are in bloom may be generated. Here, note that, alternatively, a route may be generated so as to pass through a spot popular in the SNS (for example, a spot related to a post for which a predetermined number or more of social buttons have been pressed). Further, a route may also be generated based on the degree of fatigue of the user.

Moreover, the control unit 301 may calculate a movable distance of the user based on the length of the first free time, and propose an action of the user in the first free time based on the movable distance thus calculated. The movable distance is a distance by which the user can move from the current position in the first free time, and may be calculated on the assumption that the user moves at a predetermined speed. For example, in cases where the first schedule is a work that can be performed in a public place, the control unit 301 selects, from places existing within the movable distance, a place where the first schedule is to be performed. For example, in cases Where the first schedule is a work that can be performed in a cafeteria and where a cafeteria is present within the movable distance, the control unit 301 transmits information to that effect to the user terminal 20 so that the user can move to the cafeteria and perform the work. At this time, information about a route to the cafeteria may also be transmitted.

Further, for example, in cases where the first schedule is a work including a TV conference and where the first free time is sufficiently long, the control unit 301 may set a walking route so as to enable the user to return to an original place within the first free time, and may propose a walk to the user. At this time, the walking route may be transmitted to the user terminal 20. That is, in cases where the place where the user works cannot be changed to an outside facility or the like though the free time is long enough, the user can be made to take a walk thereby to change his or her mood. In cases where the movable distance is short, i.e., in cases where the first free time is short, stretching to be performed on the spot, for example, may be recommended, instead of advising the user to move. In that case, for example, a URL (Uniform Resource Locator) can be transmitted to the user terminal 20 where a video showing how to do the stretching that can be done on the spot can be viewed.

Also, for example, in cases where the attribute of the user stored in the user information DB 311 indicates that, the user likes to drive, and in cases where the first schedule is a work that can be performed in a vehicle, the user may be recommended to drive. In this case, the control unit 301 may look for a place where parking is available within a distance that can be traveled in the first free time, and transmit a route to that place to the user terminal 20.

In addition, the control unit 301 may propose, for example, a plurality of actions so as to allow the user to select among them. For example, a proposal for selecting either stretching or walking during the first free time may be transmitted to the user terminal 20.

The control unit 301 proposes a walking route or a route to a place (alternative place) corresponding to the first schedule to the user based on the map information stored in the map information DB 314. The control unit 301 generates the walking route or the route to the alternative place in time for the start time of the first schedule, The control unit 301 transmits the route thus generated to the user terminal 20.

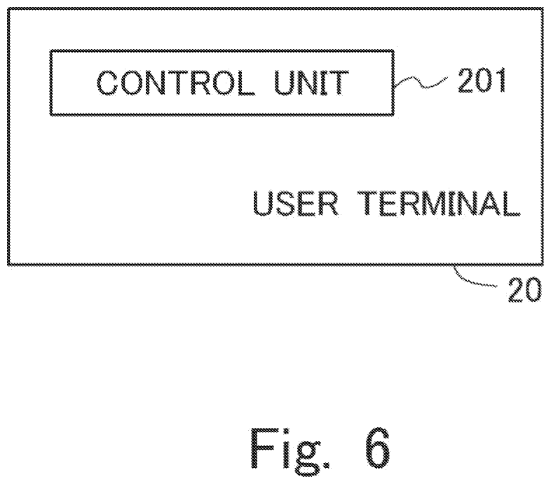

Then, the functions of the user terminal 20 will be described. FIG. 6 is a view illustrating an example of a functional configuration of the user terminal 20. The user terminal 20 includes a control unit 201 as its functional component. The processor 21 of the user terminal 20 executes the processing of the control unit 201 by a computer program on the main storage unit 22.

The control unit 201 implements an application for managing the schedules of its user (hereinafter, also referred to as a scheduler). The scheduler is, for example, an application that stores the schedules inputted or entered by the user via the input unit 24 and notifies the user that, the start time of each schedule approaches. When entering a schedule into the scheduler, the user enters a start time, an end time, a place, and a content thereof.

The control unit 201 transmits the user information and the schedule information to the server 30 at predetermined time intervals. The user information and the schedule information need not be transmitted at the same time. The user information and the schedule information are transmitted in association with identification information (user ID) unique to the user. The user information includes position information of the user terminal 20, information about the attribute of the user, and the SNS account of the user. Here, note that the position information of the user terminal 20, the information about the attribute of the user, and the SNS account of the user do not need to be all transmitted at the same time, but may be individually or separately transmitted in association with the user ID. The control unit 201 transmits an output from the position information sensor 27 to the server 30 as position information. The attribute and the SNS account of the user are entered by the user via the input unit 24. For example, when a predetermined application, which has been installed on the user terminal 20, is implemented, a screen will be displayed prompting the user to enter the attribute of the user. The control unit 201 obtains the attribute entered by the user according to the display of the screen. Also, the control unit 201 obtains the schedule information from the scheduler, and transmits it to the server 30.

Further, the control unit 201 displays, for example, the information received from the server 30 on the display 25. For example, in the case of receiving from the server 30 a proposal for an action to be performed during the first free time, the control unit 201 displays information corresponding to the proposal. For example, the control unit 201 can display statements such as “Let's go for a walk”, “Let's stretch”, or “Let's perform the first schedule in a cafeteria”, and/or it can also display a route for the walk or a route to the place where the first schedule will be performed.

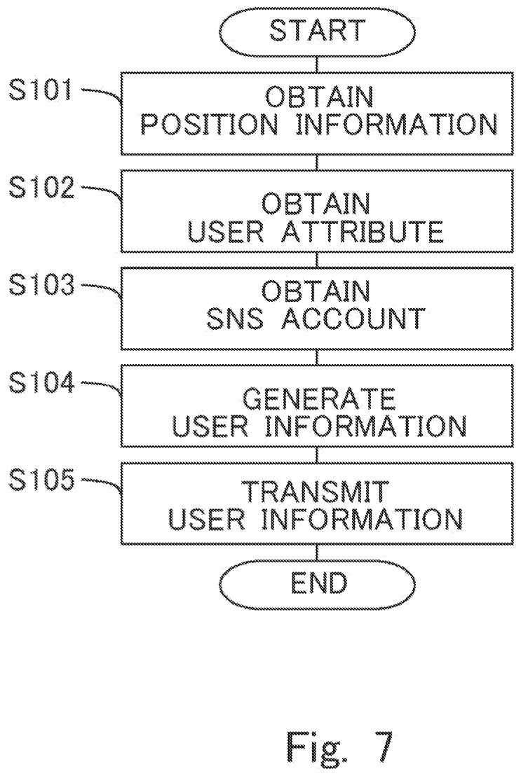

Next, processing for transmitting user information and schedule information from the user terminal 20 to the server 30 will be described. FIG. 7 is a flowchart of the processing for transmitting user information from the user terminal 20 to the server 30 according to the present, embodiment. The processing illustrated in FIG. 7 is executed at predetermined time intervals in the user terminal 20.

In step S101, the control unit 201 obtains the position information of the user terminal 20. The position information of the user terminal 20 is obtained from the position information sensor 27. In step S102, the control unit 201 obtains the attribute of the user. For example, the attribute of the user inputted or entered into the user terminal 20 by the user via the input, unit 24 has been stored in the auxiliary storage unit 23. The control unit, 201 obtains this information. In step S103, the control unit 201 obtains the SNS account of the user. The SNS account has been entered by the user via the input unit 24, and stored in the auxiliary memory unit 23.

Then, in step S104, the control unit 201 generates user information. The user information includes the position information obtained in step S101, the attribute of the user obtained in step S102, and the SNS account, obtained in step S103. The control unit 201 associates the user ID of the user with these pieces of information to generate user information. Then, in step S105, the control unit 201 transmits the user information thus generated to the server 30.

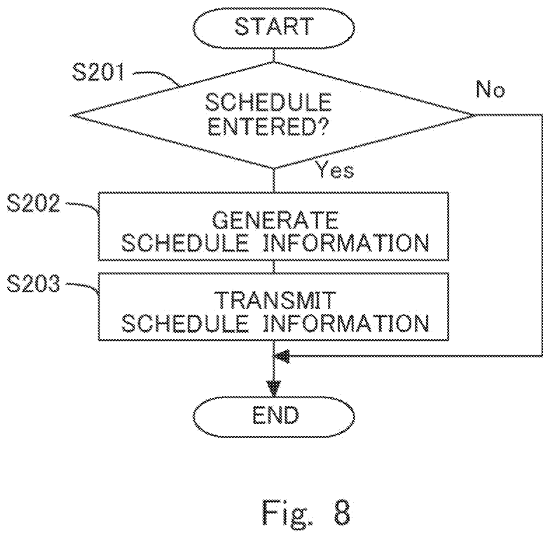

Next, FIG. 8 is a flowchart of the processing of transmitting the user information from the user terminal 20 to the server 30 according to the present embodiment. The processing illustrated in FIG. 8 is executed at predetermined time intervals in the user terminal 20.

In step S201, the control unit 201 determines whether or not the user has entered a schedule to the scheduler. The information about the schedule entered by the user is stored in the auxiliary storage unit 23 by the scheduler. When an affirmative determination is made in step S201, the processing or routine proceeds to step S202, whereas when a negative determination is made, this routine is ended. In step S202, the control unit 201 generates schedule information to be transmitted to the server 30 based on the information about the schedule entered into the scheduler. Then, in step S203, the control unit 201 transmits the schedule information to the server 30. Therefore, according to this routine, every time a schedule is entered into the scheduler, the schedule information thereof is transmitted to the server 30.

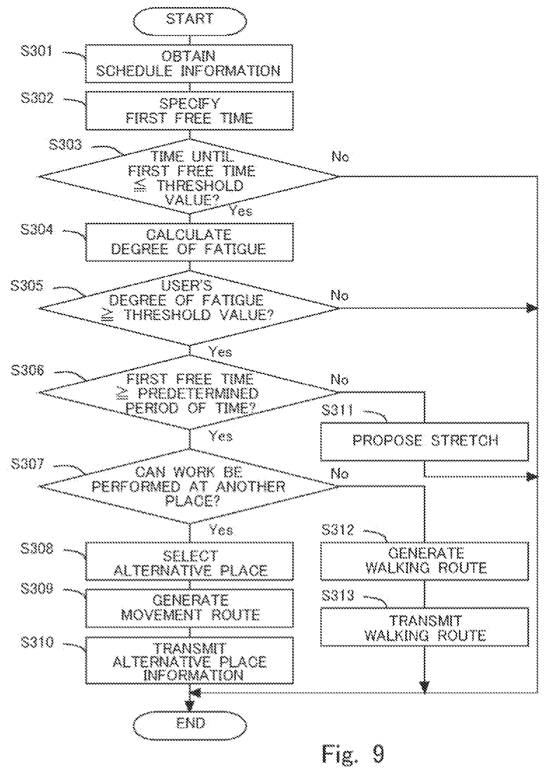

Hereafter, a description will be given of processing in which the server 30 transmits a proposal (action proposal) for an action of the user to the user terminal 20. FIG. 9 is a flowchart of the processing in which the server 30 transmits an action proposal to the user terminal 20 according to the present embodiment The processing illustrated in FIG. 9 is executed at predetermined time intervals in the server 30. Note that the following description will be made on the assumption that the user information and the schedule information have already been stored in the user information DB 311 and the schedule information DB 312, respectively. This routine is executed for each user. As a prerequisite, this routine may be executed only on days when the user needs to change his or her mood, such as business days of work.

In step S301, the control unit 301 obtains the schedule information from the schedule information DB. Then, in step S302, the control unit 301 specifies a first free time. The control unit 301 accesses the schedule information DB 312 to specify the first free time based on the end time of the schedule being performed at the moment and the start time of the next schedule. At this time, the end time of the old schedule is specified as the start time of the first free time, and the start time of the first schedule is specified as the end time of the first free time.

In step S303, the control unit 301 determines whether or not a period of time from the current time to the start time of the first free time is equal to or less than a threshold value. The threshold value is a period of time suitable for proposing an action to be performed in the first free time to the user. The threshold value may be 0. That is, the control unit 301 may determine whether or not the cu rent time is the start time of the first free time. When an affirmative determination is made in step S303, the processing or routine proceeds to step S304, whereas when a negative determination is made, this routine is ended.

In step S304, the control unit 301 calculates the degree of fatigue of the user based on the schedule information received. The control unit 301 quantifies the degree of fatigue of the user based on, for example, the length of the old schedule immediately before the first free time. For example, the degree of fatigue of the user may be calculated such that the longer the period of time of the old schedule, the greater the degree of fatigue of the user becomes. Also, as an alternative method, the control unit 301 may calculate the degree of fatigue of the user based on the performance of the work of the user in the old schedule. For example, the degree of fatigue of the user may be calculated to be higher according to the rate of decrease in the number of characters inputted or entered per unit time into the user terminal 20. Since the number of input, characters varies depending on the content (or nature) of the work, the method of calculating the degree of fatigue may be changed depending on the content of the work. In addition, as another alternative method, the degree of fatigue may be calculated based on the content of the old schedule. For example, in cases where the old schedule is a schedule about entertainment, the degree of fatigue may be calculated to be low because there is no need for a change of mood.

In step S305, the control unit 301 determines whether or not the degree of fatigue of the user is equal to or greater than a threshold value. The threshold value is set in advance as a lower limit value of the degree of fatigue at or above which a proposal is made for performing an action in the free time. The threshold value may be set by the server 30, or may be registered in advance by the user via the user terminal 20. When an affirmative determination is made in step S305, the processing or routine proceeds to step S306, whereas when a negative determination is made, this routine is ended.

In step S306, the control unit 301 determines whether or not the first free time is equal to or longer than a predetermined period of time. The predetermined period of time is set based on, for example, the time required for the user to take a walk. The first free time is calculated by the control unit 301 based on the start time of the first schedule and the end time of the old schedule, which have been entered in the schedule information DB 312. When a negative determination is made in step S306, it is not possible to propose a walk, and hence it is proposed that the user perform a stretch that can be done on the spot. That is, when a negative determination is made in step S306, the processing proceeds to step S311, where the control unit 301 transmits information for proposing a stretch to the user terminal 20. At this time, information about a stretching method may also be transmitted. On the other hand, when an affirmative determination is made in step S306, the processing proceeds to step S307.

In step S307, the control unit 301 determines whether or not the work performed in the first schedule can be performed in another place. At this time, the control unit 301 determines whether or not it is possible to work at another place (restaurant or the like) depending on the content (or nature) of the work. The control unit 301 determines this based on the content of the work stored in the schedule information DB 312. For example, in cases where the content of the work includes a TV conference or a WEB conference, it is determined that the work cannot, be performed at another place. When an affirmative determination is made in step S307, the processing proceeds to step S308, whereas when a negative determination is made, the processing proceeds to step S312.

In step S308, the control unit 301 selects an alternative place. The control unit 301 selects an alternative place based on information about the position of each place, the business hours and the content of executable work stored in the alternative place information DB 313, information about the start time, the end time, the place and the content corresponding to the first schedule stored in the schedule information DB 312, the position of the user stored in the user information DB 311, and the map information stored in the map information DB 314, For example, the control unit 301 selects, as an alternative place, a place existing within a distance in which the user can move during the first free time, a place that can accommodate the content of the work of the user, and a place that can be used until the end time of the first schedule. In cases where there is more than one applicable place, a place may be selected randomly, or the user may be asked which place is preferred, or the closest place may be selected, or a place may be selected which can take a route that matches the user's preference in conjunction with the next step S309.

Here, note that when selecting an alternative place, the control unit 301 may take into account the congestion state of the alternative place. For example, the congestion state of the alternative place may be obtained from a server that manages alternative places. Also, as another method, the congestion state may be obtained by obtaining an image of an alternative place and analyzing the image. Since the efficiency of work may decrease in places where there are many third parties, the control unit 301 may not select, for example, places having a degree of congestion equal to or higher than a predetermined congestion degree.

In addition, if the alternative place can be reserved upon selection thereof, the control unit 301 may also make a reservation for the alternative place. For example, information necessary for the reservation may be transmitted to a terminal of an administrator who manages the alternative place.

In step S309, the control unit 301 generates a travel or movement route. The movement route is a route from the current place or position of the user to the alternative place. The control unit 301 generates the route based on the position of the user stored in the user information DB 311, the position of the alternative place selected in the step S308, and the attribute of the user stored in the user information DB 311. the control unit 301 generates, for example, a route that matches the preference of the user. For example, in the case of a user who likes flowers, a route passing through a place where flowers are in bloom is generated. Such a place, which is likely to have a relationship with the preference of the user, can also be determined based on, for example, posting to the SNS. This posting may be a third party post. For example, a route may be generated so as to pass through a position based on its position information associated with a post for which a predetermined number or more of social buttons have been pressed. In this case, a route may be generated so as to pass through a position associated with a post for which a predetermined number or more of social buttons have been pressed, regardless of the hobby and preference of the user. In addition, the control unit 301 may generate, for example, a route so as to pass through a place included in a message posted from the SNS account of the user. Note that, as an alternative method, the control unit 301 may generate a route connecting the current position of the user to the alternative place in the shortest distance. Moreover, as another alternative method, the control unit 301 may generate a route such that the distance to be traveled or moved increases according to the length of the first free time. Further, as a further alternative method, the control unit 301 may generate a route for the user to walk a predetermined distance. The predetermined distance is set in advance as a distance necessary for the user to change his or her mood.

In step S310, the control unit 301 transmits the alternative place information including the movement route to the user terminal 20. The alternative place information includes information capable of specifying an alternative place such as a name or a position of the alternative place, a proposal to work at the alternative place and the like, in addition to the movement route. On the user terminal 20 that has received the alternative place information, for example, a screen for prompting the user to move to the alternative place may be displayed, so that route guidance according to the movement route may be started when the user makes a predetermined input on the screen,

On the other hand, in step S312, the control unit 301 generates a walking route. As in step S309, the walking route is generated by the control unit 301 based on the position of the user stored in the user information DB311 and the attribute of the user stored in the user information DB311. The end point of the walking route may be a place corresponding to the first schedule, or may be the same point as a departure point, or place. Then, in step S313, the control unit 301 transmits the walking route thus generated to the user terminal 20. On the user terminal 20 that has received the walking route, for example, a screen for prompting the user to walk is displayed, and when the user makes a predetermined input on the screen, route guidance according to the walking route is started.

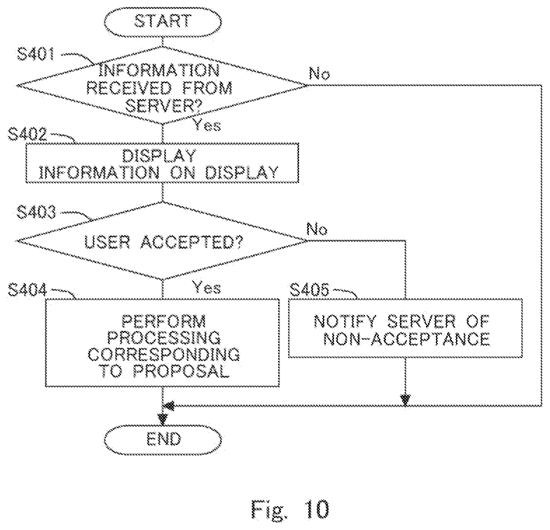

Next, FIG. 10 is a flowchart of processing executed by the user terminal 20 in the first free time according to the present embodiment. The processing illustrated in FIG. 10 is executed at, predetermined time intervals in the user terminal 20.

In step S401, the control unit 201 determines whether or not, information about the first free time has been received from the server 30. The information about the first free time is any one of the alternative place information transmitted in step S310 of FIG. 9, the information about the proposal for stretching transmitted in step S311, and the information about the walling route transmitted in step S313. When an affirmative determination is made in step S401, the processing proceeds to step S402, whereas when a negative determination is made, this routine is ended. In step S402, the control unit 201 displays the information received from the server 30 on the display 25, and also displays, for example, a radio button for allowing the user to select whether or not to accept, the proposal from the server 30.

In step S403, the control unit 201 determines whether or not the user has accepted the proposal from the server 30. The control unit 201 determines, based on the radio button pressed by the user, whether or not the user has accepted the proposal from the server 30. When an affirmative determination is made in step S403, the processing proceeds to step S404, whereas when a negative determination is made, the processing proceeds to step S405.

In step S104, the control unit 201 executes processing corresponding to the proposal received from the server 30. For example, in cases where the proposal received from the server 30 includes a movement route or a walking route to the alternative place, route guidance, may be started. Also, for example, in cases where the proposal received from the server 30 includes information about a method of stretching, the information about the method of stretching may be displayed on the display 25. On the other hand, in step S405, the control unit 201 notifies the server 30 that the proposal from the server 30 is not accepted.

Here, note that the server 30, which has received from the user terminal 20 a response indicating that the proposal from the server 30 is not accepted, may perform, for example, the processing of canceling a reservation if the reservation for an alternative place has been made. In addition, the server 30, which has received this notification, may also transmit an alternative proposal to the user terminal 20. For example, when the user does not accept an alternative place proposed by the server 30, another alternative place may be proposed, or a walk may be proposed, or a stretch may be proposed. Also, for example, in cases where the user does not accept the walk proposed by the server 30, another walking course may be proposed, or a stretch may be proposed

As described above, according to the present embodiment, it is possible to change the mood of a user by prompting the user to perform an action in a free time of his or her schedule. In addition, in cases where the place where the user works can be moved, the user can be encouraged to move the place where he or she works, which also helps change the user's mood.

### Other Embodiments

The above-described embodiment is merely an example, but the present disclosure can be implemented with appropriate modifications without departing from the spirit thereof.

The processing and/or means (devices, units, etc.) described in the present disclosure can be freely combined and implemented as long as no technical contradiction occurs.

The processing described as being performed by one device or unit may be shared and performed by a plurality of devices or units. Alternatively, the processing described as being performed by different devices or units may be performed by one device or unit. In a computer system, a hardware configuration (server configuration) for realizing each function thereof can be changed in a flexible manner. For example, the user terminal 20 may include all or some of the functions of the server 30.

The present disclosure can also be realized by supplying to a computer a computer program in which the functions described in the above-described embodiment are implemented, and reading out and executing the program by means of one or more processors included in the computer. Such a computer program may be provided to the computer by a non-transitory computer readable storage medium that can be connected to a system bus of the computer, or may be provided to the computer via a network. The non-transitory computer readable storage medium includes, for example, any type of disk such as a magnetic disk (e.g., a floppy (registered trademark) disk, a hard disk drive (HDD), etc.), an optical disk (e.g., a CD-ROM, a DVD disk, a Blu-ray disk, etc.) or the like, a read only memory (ROM), a random access memory (RAM), an EPROM, an EEPROM, a magnetic card, a flash memory an optical card, or any type of medium suitable for storing electronic commands or instructions.

What is claimed is:

1. An information processing apparatus including a controller configured to perform:
obtaining schedules of a user; and
outputting, based on a content of a first schedule among the schedules of the user, a proposal for an action of the user to change his or her mood in a first free time in which the user has no schedule immediately before the first schedule.

2. The information processing apparatus according to claim 1, wherein
the controller performs:
determining, based on the content of the first schedule, whether or not the user is capable of performing the first schedule in a predetermined place; and
proposing to move to the predetermined place in the first free time, as the proposal for the action of the user to change his or her mood, in cases where the user is the user is capable of performing the first schedule in the predetermined place.

3. The information processing apparatus according to claim 1, wherein
the controller performs:
determining, based on the content of the first schedule, whether or not the user is capable of performing the first schedule in a predetermined place; and
proposing to exercise in the first free time as the proposal for the action of the user to change his or her mood, in cases where the user is not capable of performing the first schedule in the predetermined place.

4. The information processing apparatus according to claim 3, wherein
the controller performs:
proposing to walk as the proposal to exercise.

5. The information processing apparatus according to claim 2, wherein
the controller performs:
obtaining information about an attribute of the user;
generating a movement route based on the attribute of the user; and
outputting the movement route.

6. The information processing apparatus according to claim 5, wherein
the controller performs:
obtaining the attribute of the user based on a message posted by the user to a social networking service.

7. The information processing apparatus according to claim 1, wherein
the controller performs:
proposing to perform an exercise without movement as the proposal for the action of the user to change his or her mood in the first free time when the length of the first free time is less than a predetermined period of time.

8. The information processing apparatus according to claim 1, wherein
the controller performs:
detecting a parameter correlated with a degree of fatigue of the user; and
outputting the proposal for the action of the user to change his or her mood in the first free time when the degree of fatigue of the user is equal to or greater than a threshold value.

9. An information processing method for causing a computer to perform:
obtaining schedules of a user; and
outputting, based on a content of a first schedule among the schedules of the user, a proposal for an action of the user to change his or her mood in a first free time in which the user has no schedule immediately before the first schedule.

10. The information processing method according to claim 9, wherein
the computer is caused to perform:
determining, based on the content of the first schedule, whether or not the user is capable of performing the first schedule in a predetermined place; and
proposing to move to the predetermined place in the first free time, as the proposal for the action of the user to change his or her mood, in cases where the user is capable of performing the first schedule in the predetermined place.

11. The information processing method according to claim 9, wherein
the computer is caused to perform:
determining, based on the content of the first schedule, whether or not the user is capable of performing the first schedule in a predetermined place; and
proposing to exercise in the first free time as the proposal for the action of the user to change his or her mood, in cases where the user is not capable of performing the first schedule in the predetermined place.

12. The information processing method according to claim 11, wherein
the computer is caused to perform:
proposing to walk as the proposal to exercise.

13. The information processing method according to claim 11, wherein
the computer is caused to perform:
obtaining information about an attribute of the user;
generating a movement route based on the attribute of the user; and outputting the movement route.

14. The information processing method according to claim 13, wherein
the computer is caused to perform:
obtaining the attribute of the user based on inn a message posted by the user to a social networking service.

15. The information processing method according to claim 9 wherein
the computer is caused to perform:
proposing to perform an exercise without movement as the proposal for the action of the user to change his or her mood in the first free time when the length of the first free time is less than a predetermined period of time.

16. The information processing method according to claim 9, wherein
the computer is caused to perform:
detecting a parameter correlated with a degree of fatigue of the user;
outputting the proposal for the action of the user to change his or her mood in the first free time when the degree of fatigue of the user is equal to or greater than a threshold value.

17. A system including a server configured to perform;
obtaining schedules of a user; and
transmitting to a terminal of the user, based on a content of a first schedule among the schedules of the user, a proposal for an action of the user to change his or her mood in a first free time in which the user has no schedule immediately before the first schedule.

18. The system according to claim 17, wherein
the server is configured to perform:
determining, based on the content of the first schedule, whether or not the user is capable of performing the first schedule in a predetermined place; and
proposing to move to the predetermined place in the first free time, as the proposal for the action of the user to change his or her mood, in cases where the user is the user is capable of performing the first schedule in the predetermined place.

19. The system according to claim 17, wherein the server is configured to perform:
determining, based on the content of the first schedule, whether or not the user is capable of performing the first schedule in a predetermined place; and
proposing to exercise in the first free time as the proposal for the action of the user to change his or her mood, in cases where the user is not capable of performing the first schedule in the predetermined place.

20. The system according to claim 17, wherein
the server is configured to perform:
proposing to perform an exercise without movement as the proposal for the action of the user to change his or her mood in the first free time when the length of the first free time is less than a predetermined period of time.
